(19) United States
(12) Patent Application Publication
(10) Pub. No.: US 2025/0099121 A1
(43) Pub. Date: Mar. 27, 2025

(54) THROMBECTOMY APPARATUS

(71) Applicant: Boston Scientific Scimed, Inc., Maple Grove, MN (US)

(72) Inventors: Timothy Lawrence Rubesch, Blaine, MN (US); Katherine Gingras, Rosemount, MN (US); Winnie Tang, Santa Clara, CA (US); Paul F. Chouinard, Maple Grove, MN (US); James Burke Lynch, Minneapolis, MN (US); Jayson Delos Santos, Alamo, CA (US); Roy Leguidleguid, Union City, CA (US); Cecilia Nelson, San Jose, CA (US); Clifford Van, Santa Clara, CA (US)

(73) Assignee: Boston Scientific Scimed, Inc., Maple Grove, MN (US)

(21) Appl. No.: 18/893,248

(22) Filed: Sep. 23, 2024

Related U.S. Application Data
(60) Provisional application No. 63/540,611, filed on Sep. 26, 2023.

(51) Int. Cl. A61B 17/221

(52) U.S. Cl. CPC A61B 17/221; A61B 2017/2215

(57) ABSTRACT

A thrombectomy apparatus includes a catheter having a funnel is secured to a distal end and extending distally therefrom. The funnel includes a braided inner layer, a braided outer layer, and a reinforcing structure disposed between the braided inner layer and the braided outer layer, the reinforcing structure including a plurality of arched wires extending between the braided inner layer and the braided outer layer. A tractor is adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally. An elongate member extends through the lumen and is secured to an end of the tractor disposed within the lumen.

### CROSS-REFERENCE TO RELATED APPLICATIONS

This application claims the benefit of priority under 35 U.S.C. § 119 of U.S. Provisional Application No. 63/540,611, filed Sep. 26, 2023, the entire disclosure of which is hereby incorporated by reference.

### TECHNICAL FIELD

The present disclosure relates generally to medical devices. More particularly, the present disclosure pertains to medical devices for performing thrombectomy procedures.

### BACKGROUND

A wide variety of intracorporeal medical devices have been developed for medical use, for example, intravascular use. Some of these devices include thrombectomy apparatuses. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for thrombectomy apparatuses. An example may be found in a thrombectomy apparatus for removing a clot from a vessel. The thrombectomy apparatus includes a catheter having a distal region including a distal end, the catheter defining a lumen extending proximally from the distal end, the catheter having an outer surface. A funnel is secured to the distal end and extending distally therefrom, the funnel including a braided inner layer, a braided outer layer, and a reinforcing structure disposed between the braided inner layer and the braided outer layer, the reinforcing structure including one or more arched wires extending between the braided inner layer and the braided outer layer, wherein a first end and a second end of each arched wire are each secured from movement. A tractor is adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen. An elongate member extends through the lumen and is secured to an end of the tractor disposed within the lumen.

Alternatively or additionally, the tractor may be adapted to engage the clot and pull the clot proximally into the funnel as the tractor inverts over the distal end of the funnel.

Alternatively or additionally, the tractor may include a flexible tube having a plurality of loops that lie flat before reaching the distal end of the catheter and after passing the distal end of the catheter, but temporarily extend radially outwardly as the tractor inverts over the distal end of the catheter, the plurality of loops temporarily extending radially outward as the tractor inverts over the distal end of the catheter engage the clot and pull the clot proximally.

Alternatively or additionally, the one or more arched wires may be formed into a closed loop funnel braid.

Alternatively or additionally, the first end and the second end of each of the one or more arched wires may be welded to another arched wire.

Alternatively or additionally, the closed loop funnel braid may be formed from a plurality of wires having a wire diameter greater than a filament diameter for the one or more filaments forming the braided member.

Alternatively or additionally, the plurality of wires forming the closed loop funnel braid may have a wire diameter in a range of 0.004 inches to 0.011 inches.

Alternatively or additionally, the reinforcing structure may further include a tubular base member and a plurality of fingers extending distally from the tubular base member, where the first ends and the second ends of each of the one or more arched wires may be secured relative to corresponding fingers of the plurality of fingers.

Another example may be found in a thrombectomy apparatus for removing a clot from a vessel. The thrombectomy apparatus includes a catheter having a distal region including a distal end, the catheter defining a lumen extending proximally from the distal end, the catheter having an outer surface. A funnel is secured to the distal end and extends distally therefrom. The funnel includes a braided member with a braided inner layer and a braided outer layer. The funnel includes a reinforcing structure that extends between the braided inner layer and the braided outer layer and includes one or more arched wires. A tractor is adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen. The reinforcing structure is adapted to engage the braided member when the braided member is subject to axial compression as a result of the tractor being pulled proximally over a distal end of the funnel.

Alternatively or additionally, at least some of the one or more arched wires have ends that may be constrained from movement.

Alternatively or additionally, the one or more arched wires may be formed into a closed loop funnel braid.

Alternatively or additionally, the ends of each of the one or more arched wires may be welded to another arched wire.

Alternatively or additionally, the closed loop funnel braid may be formed from a plurality of wires having a wire diameter greater than a filament diameter for the one or more filaments forming the braided member.

Alternatively or additionally, the plurality of wires forming the closed loop funnel braid may have a wire diameter in a range of 0.004 inches to 0.011 inches.

Alternatively or additionally, the reinforcing structure may further include a tubular base member and a plurality of fingers extending distally from the tubular base member, where the first ends and the second ends of each of the one or more arched wires may be secured relative to corresponding fingers of the plurality of fingers.

Another example may be found in a thrombectomy apparatus for removing a clot from a vessel. The thrombectomy apparatus includes a catheter having a distal region including a distal end, the catheter defining a lumen extending proximally from the distal end, the catheter having an outer surface. A funnel is secured to the distal end and extends distally therefrom. The funnel includes a braided member having a braided inner layer and a braided outer layer. The funnel also includes a reinforcing structure extending between the braided inner layer and the braided outer layer, the reinforcing structure including one or more arched wires adapted to engage the braided member when the braided member is subjected to an axial compression force. A tractor is adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen.

Alternatively or additionally, at least some of the one or more arched wires may have ends that are constrained from movement.

Alternatively or additionally, the one or more arched wires may be formed into a closed loop funnel braid.

Alternatively or additionally, the ends of each of the one or more arched wires may be welded to another arched wire.

Alternatively or additionally, the reinforcing structure may further include a tubular base member and a plurality of fingers extending distally from the tubular base member, where the first ends and the second ends of each of the one or more arched wires may be secured relative to corresponding fingers of the plurality of fingers.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an illustrative thrombectomy apparatus shown in a starting position proximate a clot within a blood vessel;

FIG. 2 is a schematic view of the illustrative thrombectomy apparatus of FIG. 1, shown in a finishing position after having captured the clot from within the blood vessel;

FIG. 3 is a schematic view of an illustrative tractor disposed over an illustrative funnel as part of the illustrative thrombectomy apparatus of FIG. 1;

FIG. 4 is a schematic view of the illustrative thrombectomy apparatus of FIG. 1 showing a reinforcing structure disposed within a braided member;

FIG. 5 is a schematic view of an illustrative reinforcing structure;

FIG. 6 is a schematic view of an illustrative reinforcing structure;

FIG. 7 is a schematic view of the illustrative thrombectomy apparatus of FIG. 1 shown passing through a tortuous vasculature prior to proximally withdrawing the tractor;

FIG. 8 is a schematic view of the illustrative thrombectomy apparatus of FIG. 1 shown passing through a tortuous vasculature after proximally withdrawing the tractor;

FIG. 9 is a schematic view of an illustrative reinforcing structure;

FIG. 10 is a schematic view of an illustrative reinforcing structure;

FIG. 11 is a schematic view of an illustrative reinforcing structure; and

FIG. 12 is a schematic view of an illustrative braided member including a braided outer layer and a braided inner layer, the illustrative braided member forming a funnel that is part of the illustrative thrombectomy apparatus of FIG. 1.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

Thrombectomy is a process whereby a thrombus, or clot, is removed from within a blood vessel. FIG. 1 is a schematic view of an illustrative thrombectomy apparatus 10 that is shown positioned within an example blood vessel 12. A thrombus or clot 14 is schematically shown within the blood vessel 12. The thrombectomy apparatus 10 includes a catheter 16 that extends from a proximal region 18 to a distal region 20 and includes a distal end 22. A lumen 24 extends through the catheter 16. The catheter 16 includes an outer surface 26. A funnel 28 is secured to the distal end 22 and extends distally from the distal end 22. In some instances, the funnel 28 is a braided structure including a braided inner layer and a braided outer layer, as will be shown in subsequent drawings. As will be discussed, in some instances the braided outer layer may be adapted to constrain the braided inner layer from expanding too far in a radially outward direction as a result of an axial compressive force.

A tractor 30 is adapted to extend over the outer surface 26 of the catheter 16 and the funnel 28 in an un-inverted configuration and to extend in an inverted configuration through the funnel 28 and into the lumen 24. In some instances, the tractor 30 may be adapted to invert by rolling over a distal end 32 of the funnel 28 when the tractor 30 moves proximally within the lumen 24. The thrombectomy apparatus 10 includes an elongate member 34 that extends through the lumen 24 and is operably coupled with an end 36 of the tractor 30 that is disposed within the lumen 24. In some instances, the tractor 30 is adapted to engage the clot 14 and to pull the clot 14 proximally into the funnel 28 and thus into the lumen 24 as the tractor 30 inverts over the distal end 32 of the funnel 28.

The thrombectomy apparatus 10 includes an elongate member 34 that is adapted to extend through the lumen 24 within the catheter 16. The elongate member 34 is secured to the end 36 of the tractor 30 that is disposed within the lumen 24. In some instances, the elongate member 34 may be a catheter. In some instances, the elongate member 34 may be a pull wire or other pull member that is flexible enough to bend within the lumen 24 as the catheter 16 navigates the vasculature, but strong enough to allow a user to pull the elongate member 34 proximally in order to pull the tractor 30 proximally into the funnel 28 and thus into the lumen 24.

In some instances, the thrombectomy apparatus 10 may include a sheath 38. The sheath 38 may overly the catheter 16 and the tractor 30. In some instances, the sheath 38 helps prevent the tractor 38 from engaging or otherwise contacting the walls of the blood vessel 12. In some instances, the sheath 38 may have an inner diameter sufficient to prevent the tractor 30 from engaging the walls of the blood vessel 12 while still allowing the tractor 30 to move relative to the outer surface 26 of the catheter 16 as the tractor 30 moves. In some instances, the sheath 38 or at least an inner layer or coating may be formed of a lubricious material such as but not limited to a fluoropolymer. As an example, the sheath 38 may be formed of, or may at least have an inner layer or coating that is formed of a fluoropolymer such as polytetrafluoroethylene (PTFE), commonly known as Teflon®.

In some instances, the sheath 38 may be positioned further distally relative to the funnel 28 during advancement of the thrombectomy apparatus 10 through the vasculature. In some instances, the sheath 38 may constrain the funnel 28 into a smaller diameter configuration while the thrombectomy apparatus 10 is advanced through the vasculature. Once a desired location has been reached, the sheath 38 may be withdrawn proximally a short distance in order to allow the funnel 28 to expand into a conical shape, as shown. Prior to subsequent removal of the thrombectomy apparatus 10, the sheath 38 may be advanced distally a short distance in order to once again collapse the funnel 28 into a smaller diameter configuration for navigation.

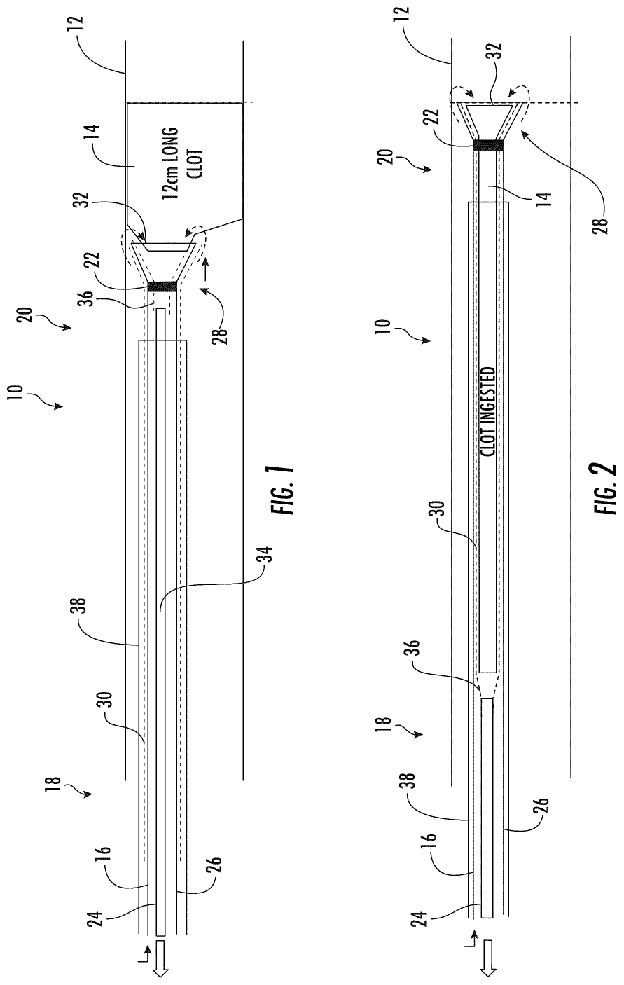

As noted, FIG. 1 shows the thrombectomy apparatus 10 positioned within the blood vessel 12, with the distal end 32 of the funnel 28 positioned adjacent or even in contact with the clot 14. By withdrawing the elongate member 34 proximally, the tractor 30 is caused to move over the distal end 32 of the funnel 28, and to pass through an interior of the funnel 28 and into the lumen 24. In some instances, as the tractor 30 moves from an un-inverted configuration proximal of the distal end 32 of the funnel 28, to an inverted configuration as the tractor 30 moves beyond the distal end 32 of the funnel 28, the tractor 30 is adapted to grab the clot 14 and to pull the clot 14 into the funnel 28 and into the lumen 24. The culmination of this may be seen in FIG. 2, which shows the thrombectomy apparatus 10 in a finishing position in which the clot 14 has been completely pulled into an interior of the thrombectomy apparatus 10. As can be seen in FIG. 2, the clot 14 is positioned within the lumen 24 and within the funnel 28. At this point, withdrawal of the thrombectomy apparatus 10 means that clot 14 will have been removed from the patient.

It will be appreciated that the clot 14 has a length when disposed within the blood vessel 12. In some instances, during the process of capturing the clot 14, the thrombectomy apparatus 10 will move distally. In some instances, the thrombectomy apparatus 10 will move distally a distance about equal to the length of the clot 14 when the clot 14 is still in the blood vessel 12. If the undisturbed clot 14 has a length of 12 centimeters, the thrombectomy apparatus 10 may move distally a distance of about 12 centimeters in the process of capturing the clot 14. In some instances, depending on the nature of the clot 14, the thrombectomy apparatus 10 may move distally a distance greater than the length of the undisturbed clot 14, for example.

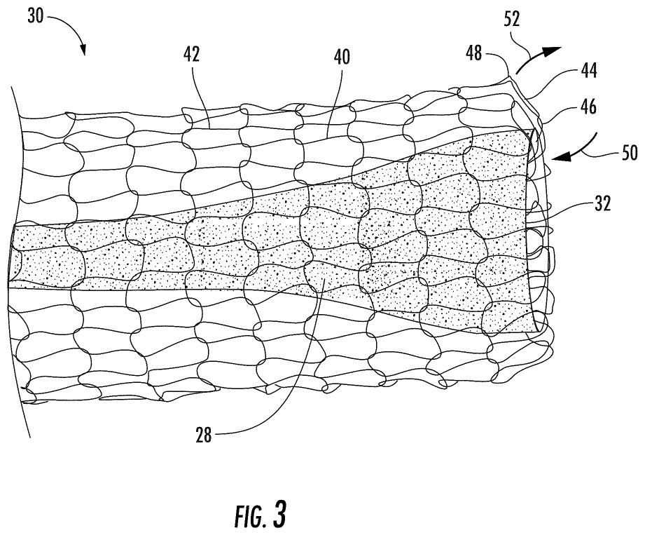

FIG. 3 is a schematic view of the tractor 30 disposed over the funnel 28. In some instances, as shown, the tractor 30 may be formed from a plurality of loops 40. In some instances, the tractor 30 may be formed by knitting one or more filaments 42 into the plurality of loops 40. A variety of different knitting patterns may be utilized, for example. It will be appreciated that the portion of the tractor 30 shown outside of the funnel 28 is in an un-inverted configuration while the portion of the tractor 30 inside the funnel 28 (not visible) is in an inverted configuration. The tractor 30 inverts from the un-inverted configuration to the inverted configuration as the tractor 30 moves around the distal end 32 of the funnel 28. With particular attention to a specific loop 44, it can be seen that the loop 44 has a leading edge 46 and a trailing edge 48. As the loop 44 pivots around the distal end 32 of the funnel 28, the leading edge 46 moves in a direction indicated by an arrow 50 while the trailing edge 46 moves in a direction indicated by an arrow 52. Accordingly, as each of the loops 44 pivot around the distal end 32 of the funnel 28, each loop 44 will temporarily extend radially outwardly as the loop 44 pivots. Otherwise, each of the loops 44 will lay flat, particularly as tension is placed on the tractor 30.

Additional details regarding the thrombectomy apparatus 10, including the catheter 16, the tractor 30 and the elongate member 34, may be found in U.S. Pat. Nos. 9,962,178; 10,835,269; US 2022/0257269 and US 2022/0287053, each of which are incorporated by reference herein. It will be appreciated that as the tractor 30 is pulled into the funnel 28 and into the lumen 24 by withdrawing the elongate member 34 proximally, and as a result the tractor 30 inverts over the distal end 32 of the funnel 28, that an axial compressive force is applied to the funnel 28 by the tractor 30. In some instances, the funnel 28 may be adapted to resist or otherwise accommodate this axial compressive force such that the funnel 28 is able to retain its tapered shape. In some instances, as will be discussed, the funnel 28 may be a braided structure. Braided structures can have a tendency to increase in diameter in response to a decrease in length, and of course the inverse is true as well. A braided structure can have a tendency to decrease in diameter in response to an increase in length. In some instances, the funnel 28 may be a braided structure having an inner braided layer and an outer braided layer.

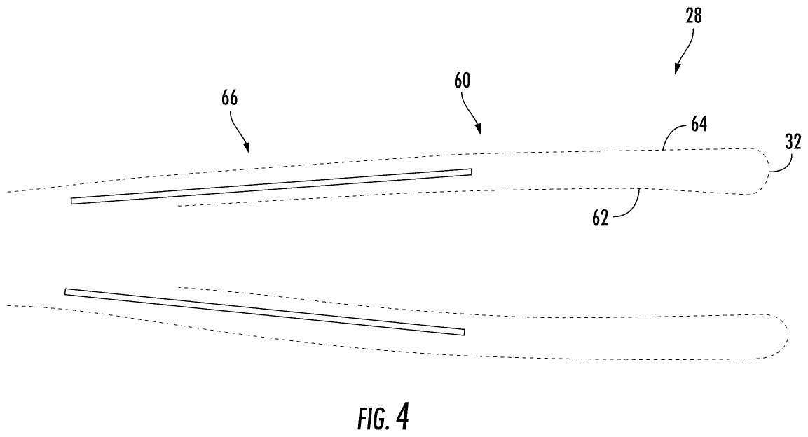

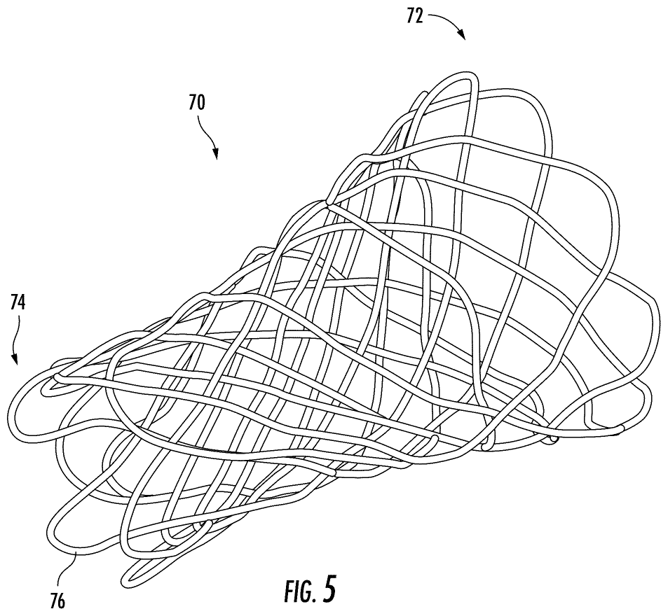

In some instances, the thrombectomy apparatus 10 may include a reinforcing structure disposed within or otherwise forming part of the funnel 28. The reinforcing structure may be adapted to help the funnel 28 retain a conical shape even when the funnel 28 is undergoing an axial compressive force as a result of the tractor 30 being pulled proximally over the distal end 32 of the funnel 28. FIG. 4 is a schematic view showing several features of the funnel 28. The funnel 28 includes a braided member 60 that is folded over on itself to have a braided inner layer 62 and a braided outer layer 64. A reinforcing structure 66 may be seen as being disposed between the braided inner layer 62 and the braided outer layer 64.

In some instances, as shown, the funnel 28 may be considered as having a tapered profile tapering from a maximum diameter at the distal end 32 of the funnel 28 to a minimal diameter at an opposing proximal end. In some instances, the reinforcing structure 66 may include a plurality of arched wires. The arched wires may be combined into a braided or woven structure in which many if not all of the free ends of each of the arched wires are welded together to form a closed structure. In some instances, the arched wires may extend between the braided inner layer 62 and the braided outer layer 64. In some instances, the arched wires may extend distally from a support structure such as a hypotube. In some instances, the arched wires may extend distally from a number of metal fingers, for example. In some instances, inclusion of the arched wires provides desired support for the braided member 60 without requiring sutures placed within the braided member 60. In some instances, inclusion of the arched wires may help the thrombectomy apparatus 10 navigate and be actuated within a tortuous vasculature while retaining a desired conical shape of the funnel 28. The reinforcing structure 66 may take multiple forms, as will be shown for example in FIGS. 5-6 and 9-11.

FIG. 5 is a schematic view of an illustrative reinforcing structure 70 that may be considered as being an example of the reinforcing structure 66 shown in FIG. 4. The reinforcing structure 70 extends from a distal end 72 to a proximal end 74. In some instances, when the reinforcing structure 70 is disposed within the braided member 60 such that the reinforcing structure 70 is disposed between the braided inner layer 62 and the braided outer layer 64, the distal end 72 may be positioned close to where the braided member 60 is folded over to form the braided inner layer 62 and the braided outer layer 64, particularly after the funnel 28 has compressed axially as a result of the tractor 30 being pulled proximally over the distal end 32 of the funnel 28. In some instances, the reinforcing structure 70 will have intersecting angles that are different from those of the braided inner layer 62 and the braided outer layer 64, and thus can conflict with the braided member 60 and thus limit relative axial compression (and radial expansion) of the braided member 60.

In some instances, the reinforcing structure 70 may be considered as being a closed loop funnel braid. The reinforcing structure 70 may be formed by weaving together one or more wires 76. In some instances, the reinforcing structure 70 may be formed from a single continuous wire that is woven into a braided structure. Each of the wires 76 may have a first end and a second end, and each of the first ends and the second ends of each of the wires 76 may be welded together to form closed loops. In some instances, some of the free ends may be welded and others of the free ends may not be welded. The reinforcing structure 70 may be formed from any number of wires 76. As shown, the reinforcing structure 70 may include a total of twelve wires 76. In some instances, the reinforcing structure 70 may be formed from eight, nine, ten or eleven wires 76. In some instances, the reinforcing structure 70 may be formed from thirteen, fourteen, fifteen or more wires 76. In some instances, the wires 76 may have a wire diameter ranging from 0.004 inches to 0.011 inches. In some instances, the reinforcing structure 70 may have fewer wires 76 when each of the wires 76 are relatively larger in diameter, and may have more wires 76 when each of the wires 76 are relatively smaller in diameter. In some instances, the wires 76 may all have the same wire diameter. In some instances, the wires 76 may not all have the same wire diameter, and thus the wire diameter may vary from wire to wire. The wires 76 may be formed of any suitable metal. In some instances, the wires 76 may be formed of nitinol, for example.

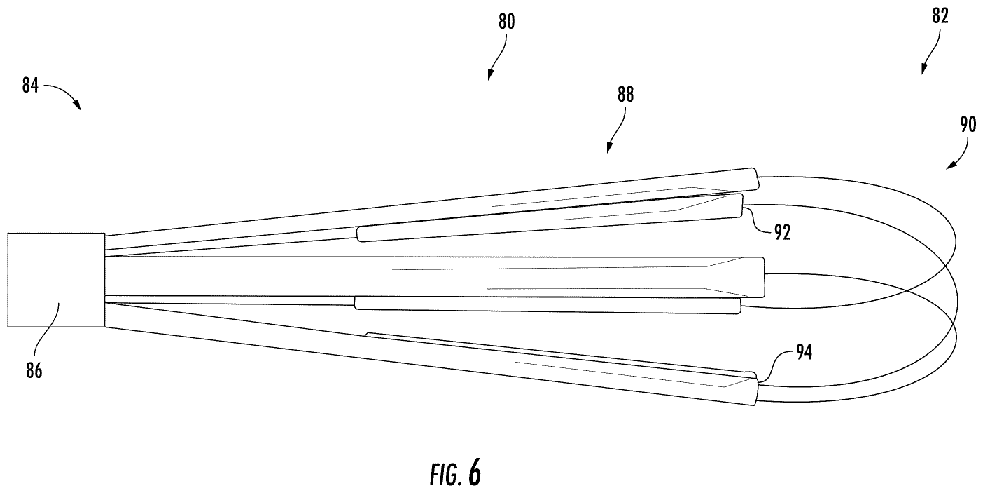

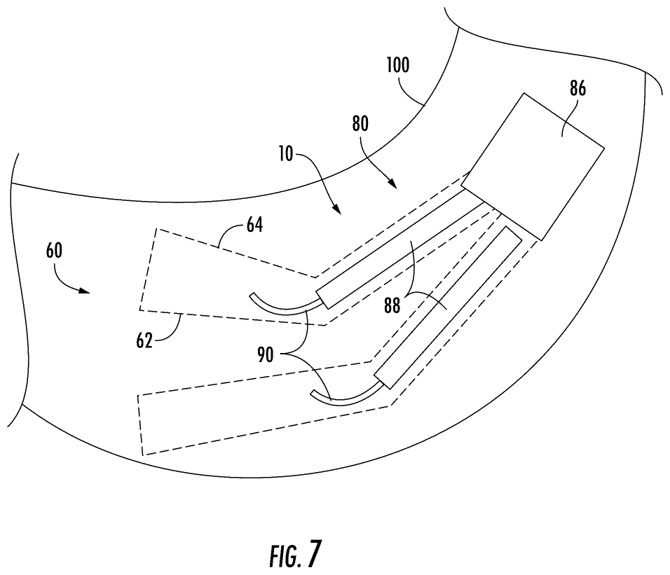

FIG. 6 is a schematic view of an illustrative reinforcing structure 80 that may be considered as being an example of the reinforcing structure 66 shown in FIG. 4. The reinforcing structure 80 extends from a distal end 82 to a proximal end 84. The reinforcing structure 80 includes a tubular base member 86 and a plurality of fingers 88 extending distally from the tubular base member 86. In some instances, for example, the reinforcing structure 80 may be formed by cutting longitudinal slits into a hypotube to form a number of finger blanks. The longitudinal slits may be laser-cut, for example. Each of the finger blanks can be folded back over itself, and each of the finger blanks may be splayed somewhat to form the plurality of fingers 88. In some instances, the fingers 88 are not folded over on themselves, and have a length determined by the length of the hypotube from which the fingers 88 are cut.

The reinforcing structure 80 includes a number of arched wires 90 that each extend from a distal end of one finger 88 to a distal end of another finger 88. How many arched wires 90 are included may be a function of how many fingers 88 there are. Each arched wire 90 may extend from a first finger 88 to a second finger 88 that is positioned circumferentially about 180 degrees from the first finger 88. Other arrangements are also contemplated. In some instances, each arched wire 90 may have a first end 92 and a second end 94. The first end 92 may be secured to a first finger 88 and the second end 94 may be secured to a second finger 88. As an example, the arched wires 90 may be welded to the distal ends of the fingers 88. Adhesives may also be used, for example. In some instances, the fingers 88 may be shorter than shown, and the arched wires 90 may be longer. In some instances, the fingers 88 may be eliminated, and the arched wires 90 may extend distally directly from the tubular base member 86. The arched wires 88 may be formed of any suitable metal. In some instances, the arched wires 88 may be formed of nitinol.

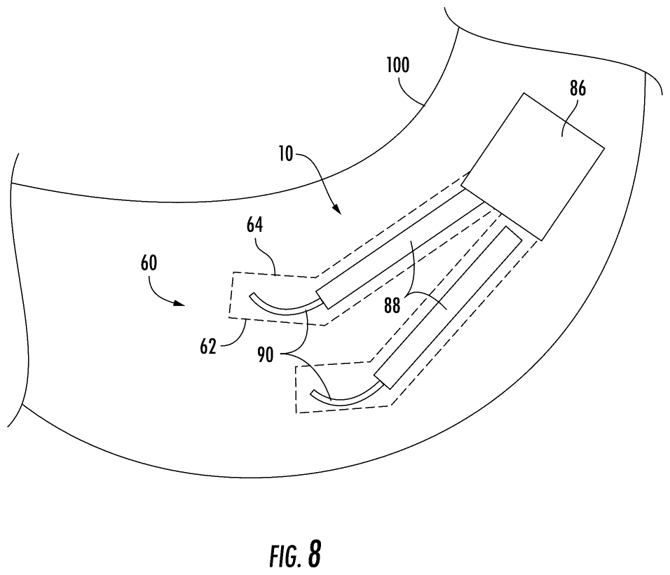

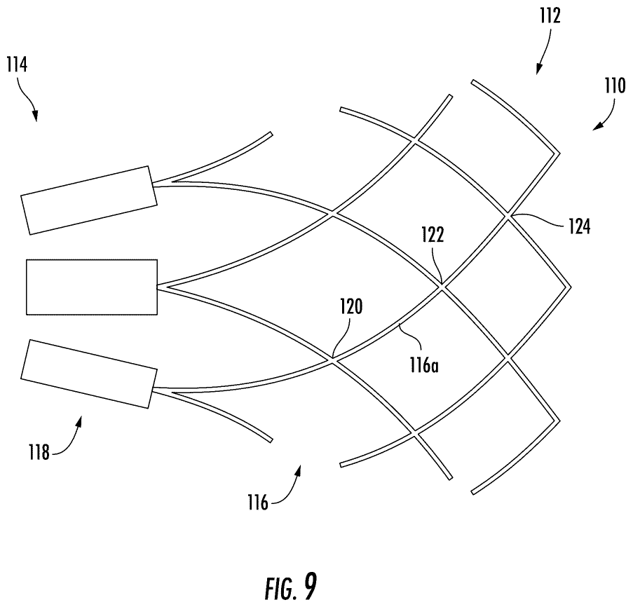

FIGS. 7 and 8 provide a schematic example of how inclusion of the arched wires 90 may facilitate use of a thrombectomy apparatus within a tortuous vasculature, and may help the funnel 28 retain its desired shape. FIGS. 7 and 8 shows the thrombectomy apparatus 10 within a curved portion of vasculature 100. The thrombectomy apparatus 10 includes the reinforcing structure 80. In FIG. 7, the tractor 30 has not yet been pulled proximally, so the braided member 60, including the braided inner layer 62 and the braided outer layer 64, extends distally beyond a distal end of the arched wires 90. In FIG. 8, the tractor 30 has been pulled proximally. As a result, the braided member 60, including the braided inner layer 62 and the braided outer layer 64, has compressed to the point that the braided member 60 contacts the arched wires 90. The arched wires 90 help to keep the fingers 88 centered between the braided inner layer 62 and the braided outer layer 64.

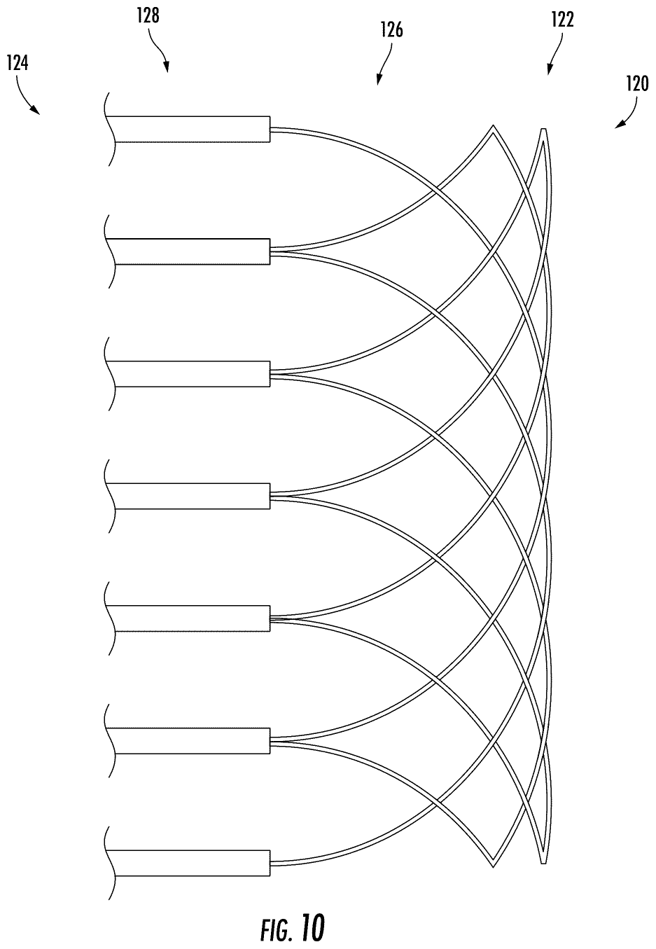

FIG. 9 is a schematic view of an illustrative reinforcing structure 110 that may be considered as being an example of the reinforcing structure 66. It will be appreciated that FIG. 9 is a two-dimensional rendering of a three-dimensional structure. The reinforcing structure 110 extends from a distal end 112 to a proximal end 114. The reinforcing structure 110 will have a maximum diameter at or near the distal end 112 and a minimum diameter at or near the proximal end 114, and may taper therebetween. The reinforcing structure 110 includes a number of arched wires 116 that extend distally from each of a number of fingers 118. In some instances, each arched wire 116 may have a first end that is secured to one of the fingers 118 and a second end that is secured to another of the fingers 118. In some instances, the first end of each arched wire 116 may be secured to one of the fingers 118 and the opposing end of each arched wire 116 may instead be secured to another of the arched wires 116. The arched wires 116 may be laid one on top of another, or the arched wires 116 may be braided together in an under-over pattern. While the reinforcing structure 110 is shown as including the fingers 118, in some instances, the fingers 118 may be excluded, and the arched wires 116 may attach to the catheter (such as the catheter 16), for example.

In some instances, the arched wires 116 may intersect each other. Looking at a particular wire 116a, the wire 116a intersects other arched wires 116 at a first intersection 120, a second intersection 122, a third intersection 124, and so on. In some instances, the wire 116a may be free to move relative to the other wires 116 at each of the first intersection 120, the second intersection 122, the third intersection 124 and so on. In some instances, the wire 116a may be secured to one or more of the other wires 116 at one or more of the first intersection 120, the second intersection 122, the third intersection 124 and so on. In some instances, the wire 116a may be secured to each of the other wires 116 at each of the first intersection 120, the second intersection 122, the third intersection 124 and so on. The arched wires 116 may be formed of any suitable metal. In some instances, the arched wires 116 may be nitinol. The arched wires 116 may have a wire diameter in a range of 0.002 inches to 0.005 inches.

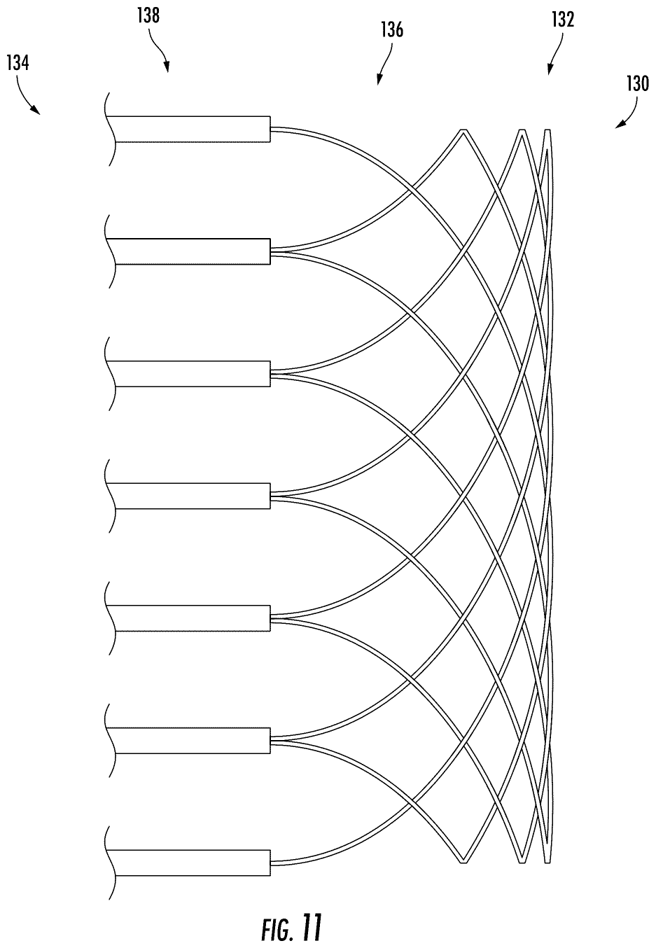

FIG. 10 is a schematic view of an illustrative reinforcing structure 120 that may be considered as being an example of the reinforcing structure 66. It will be appreciated that FIG. 10 is a two-dimensional rendering of a three-dimensional structure. The reinforcing structure 120 extends from a distal end 122 to a proximal end 124. The reinforcing structure 120 will have a maximum diameter at or near the distal end 122 and a minimum diameter at or near the proximal end 124, and may taper therebetween. The reinforcing structure 120 includes a number of arched wires 126 that extend distally from each of a number of fingers 128. As shown, each of the arched wires 126 have a first end secured to a first finger 128 and a second end secured to a second finger 128. In some instances, the arched wires 126 cross other arched wires 126 in a number of intersections. Some of these intersections may involve one arched wire 126 crossing over another arched wire 126 without any securement therebetween. Some of these intersections may include one arched wire 126 welded or otherwise secured to another arched wire 126.

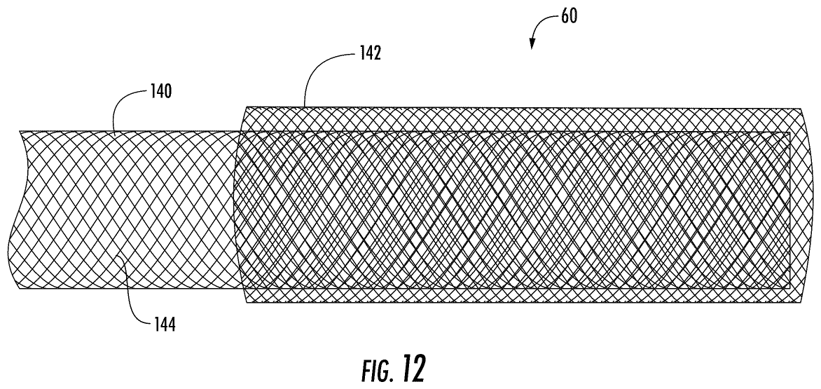

FIG. 11 is a schematic view of an illustrative reinforcing structure 130 that may be considered as being an example of the reinforcing structure 66. It will be appreciated that FIG. 11 is a two-dimensional rendering of a three-dimensional structure. The reinforcing structure 130 extends from a distal end 132 to a proximal end 134. The reinforcing structure 130 will have a maximum diameter at or near the distal end 132 and a minimum diameter at or near the proximal end 134, and may taper therebetween. The reinforcing structure 130 includes a number of arched wires 136 that extend distally from each of a number of fingers 138. As shown, each of the arched wires 136 have a first end secured to a first finger 138 and a second end secured to a second finger 138. In some instances, the arched wires 136 cross other arched wires 136 in a number of intersections. Some of these intersections may involve one arched wire 136 crossing over another arched wire 136 without any securement therebetween. Some of these intersections may include one arched wire 136 welded or otherwise secured to another arched wire 136.

FIG. 12 is a schematic view of the braided member 60 before being constrained to form the funnel 28. As can be seen, the braided member 60 includes a first portion 140 that will form the braided inner layer 62 and a second portion 142 that will form the braided outer layer 64. The second portion 144 has been doubled over the first portion 142. During the manufacturing process, first the braided inner layer 62 and then the braided outer layer 64 will be constrained into the conical shape of the funnel 28. In some instances, the first portion 140 and the second portion 142 of the braided member 60 may be formed by braiding the braided member 60 from one or more filaments 144, with an similar braid angle throughout the braided member 60. It will be appreciated that the braid angle will change considerably when the braided member 60 is compressed down into the funnel 28. In some instances, the one or more filaments 144 may have a wire diameter that is smaller than the wires 76 used to form the reinforcing structure 70, the reinforcing structure 80, the reinforcing structure 110, the reinforcing structure 120 or the reinforcing structure 130. The one or more filaments 144 may have a wire diameter that is in a range of 0.002 inches to 0.005 inches, for example.

The materials that can be used for the various components of the devices and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent and/or elements or components thereof. In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A thrombectomy apparatus for removing a clot from a vessel, the thrombectomy apparatus comprising:
a catheter having a distal region including a distal end, the catheter defining a lumen extending proximally from the distal end, the catheter having an outer surface;
a funnel secured to the distal end and extending distally therefrom, the funnel including:
a braided inner layer;
a braided outer layer; and
a reinforcing structure disposed between the braided inner layer and the braided outer layer, the reinforcing structure comprising one or more arched wires extending between the braided inner layer and the braided outer layer,
wherein a first end and a second end of each arched wire are each secured from movement;
a tractor adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen; and
an elongate member extending through the lumen and secured to an end of the tractor disposed within the lumen.

2. The thrombectomy apparatus of claim 1, wherein the tractor is adapted to engage the clot and pull the clot proximally into the funnel as the tractor inverts over the distal end of the funnel.

3. The thrombectomy apparatus of claim 2, wherein the tractor comprises a flexible tube comprising a plurality of loops that lie flat before reaching the distal end of the catheter and after passing the distal end of the catheter, but temporarily extend radially outwardly as the tractor inverts over the distal end of the catheter, the plurality of loops temporarily extending radially outward as the tractor inverts over the distal end of the catheter engage the clot and pull the clot proximally.

4. The thrombectomy apparatus of claim 1, wherein the one or more arched wires are formed into a closed loop funnel braid.

5. The thrombectomy apparatus of claim 4, wherein the first end and the second end of each of the one or more arched wires are welded to another arched wire.

6. The thrombectomy apparatus of claim 4, wherein the closed loop funnel braid is formed from a plurality of wires having a wire diameter greater than a filament diameter for the one or more filaments forming the braided member.

7. The thrombectomy apparatus of claim 6, wherein the plurality of wires forming the closed loop funnel braid have a wire diameter in a range of 0.004 inches to 0.011 inches.

8. The thrombectomy apparatus of claim 1, wherein the reinforcing structure further comprises:
a tubular base member; and
a plurality of fingers extending distally from the tubular base member;
wherein the first ends and the second ends of each of the one or more arched wires are secured relative to corresponding fingers of the plurality of fingers.

9. A thrombectomy apparatus for removing a clot from a vessel, the thrombectomy apparatus comprising:
a catheter having a distal region including a distal end, the catheter defining a lumen extending proximally from the distal end, the catheter having an outer surface;
a funnel secured to the distal end and extending distally therefrom, the funnel including:
a braided member including:
a braided inner layer; and
a braided outer layer; and
a reinforcing structure extending between the braided inner layer and the braided outer layer, the reinforcing structure comprising one or more arched wires; and
a tractor adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen;
wherein the reinforcing structure is adapted to engage the braided member when the braided member is subject to axial compression as a result of the tractor being pulled proximally over a distal end of the funnel.

10. The thrombectomy apparatus of claim 9, wherein at least some of the one or more arched wires have ends that are constrained from movement.

11. The thrombectomy apparatus of claim 9, wherein the one or more arched wires are formed into a closed loop funnel braid.

12. The thrombectomy apparatus of claim 11, wherein the ends of each of the one or more arched wires are welded to another arched wire.

13. The thrombectomy apparatus of claim 11, wherein the closed loop funnel braid is formed from a plurality of wires having a wire diameter greater than a filament diameter for the one or more filaments forming the braided member.

14. The thrombectomy apparatus of claim 13, wherein the plurality of wires forming the closed loop funnel braid have a wire diameter in a range of 0.007 inches to 0.011 inches.

15. The thrombectomy apparatus of claim 9, wherein the reinforcing structure further comprises:
a tubular base member; and
a plurality of fingers extending distally from the tubular base member;
wherein the first ends and the second ends of each of the one or more arched wires are secured relative to corresponding fingers of the plurality of fingers.

16. A thrombectomy apparatus for removing a clot from a vessel, the thrombectomy apparatus comprising:
a catheter having a distal region including a distal end, the catheter defining a lumen extending proximally from the distal end, the catheter having an outer surface;
a funnel secured to the distal end and extending distally therefrom, the funnel including:
a braided member including:
a braided inner layer; and
a braided outer layer; and
a reinforcing structure extending between the braided inner layer and the braided outer layer, the reinforcing structure comprising one or more arched wires adapted to engage the braided member when the braided member is subjected to an axial compression force; and
a tractor adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally over the lumen.

17. The thrombectomy apparatus of claim 16, wherein at least some of the one or more arched wires have ends that are constrained from movement.

18. The thrombectomy apparatus of claim 16, wherein the one or more arched wires are formed into a closed loop funnel braid.

19. The thrombectomy apparatus of claim 18, wherein the ends of each of the one or more arched wires are welded to another arched wire.

20. The thrombectomy apparatus of claim 16, wherein the reinforcing structure further comprises:
a tubular base member; and
a plurality of fingers extending distally from the tubular base member;
wherein the first ends and the second ends of each of the one or more arched wires are secured relative to corresponding fingers of the plurality of fingers.
